(19) United States
(12) Patent Application Publication
(10) Pub. No.: US 2025/0009934 A1
(43) Pub. Date: Jan. 9, 2025

(54) ANTICOAGULANT ACELLULAR VASCULAR GRAFTS AND METHODS THEREOF

(71) Applicant: THE TEXAS A&M UNIVERSITY SYSTEM, College Station, TX (US)

(72) Inventors: Feng ZHAO, College Station, TX (US); Weilue HE, Houghton, MI (US)

(21) Appl. No.: 18/709,960

(22) PCT Filed: Nov. 11, 2022
(86) PCT No.: PCT/US2022/079698

§ 371 Date: May 14, 2024

Related U.S. Application Data
(60) Provisional application No. 63/279,239, filed on Nov. 15, 2021.

(51) Int. Cl. A61L 27/36; A61L 27/22; A61L 27/52; A61L 27/54; A61L 33/00

(52) U.S. Cl. CPC A61L 27/3633; A61L 27/222; A61L 27/3679; A61L 27/52; A61L 27/54; A61L 33/0011; A61L 2300/236; A61L 2300/42; A61L 2400/12; A61L 2430/22

(57) ABSTRACT

The present disclosure provides a vascular graft comprising i) one or more extracellular matrix compositions and ii) one or more hydrogels. In particular, the vascular grafts of the present disclosure can be functionalized and/or be completely biologic. The disclosure also provides methods of administering the vascular grafts as well as methods for forming the vascular grafts.

### CROSS-REFERENCE TO RELATED APPLICATIONS

This application claims the benefit under 35 USC § 119(e) of U.S. Provisional Application Ser. No. 63/279,239, filed on Nov. 15, 2021, the entire disclosure of which is incorporated herein by reference.

### GOVERNMENT RIGHTS

This invention was made with government support under 1R15HL145654-01 awarded by the National Institute of Health. The government has certain rights in the invention.

### BACKGROUND AND SUMMARY

Cardiovascular disease causes over 360,000 deaths per year and costs over $200 billion annually for health care and medications in the United States. In the US alone, approximately 1.4 million surgeries are performed every year for the revascularization of coronary and peripheral arteries. Currently, the standard for small-diameter vessels (inner diameter<6 mm) is autologous grafts that are obtained via a harvesting procedure associated with significant morbidities and unknown long-term complications to the patients. Additionally, healthy autologous vessels are unavailable in over 100,000 patients due to the systematic pathological changes in their vascular system or previous vein harvests.

Although there is a high demand for small-diameter vascular grafts, no tissue engineered vascular grafts (TEVGs) have achieved clinical success or can meet the urgent needs of patients. Further complications include biocompatibility challenges associated with synthetic material based grafts, stringent storage and delivery conditions, long lead time to prepare conventional TEVG if using patient specific cells, graft patency, adverse immune reactions, and infections. Therefore, there exists a need to develop new vascular grafts and methods thereof to address the challenges posed by current practices.

Accordingly, the present disclosure provides a vascular graft as well as associated methods and compositions thereof. As detailed in the present disclosure, several benefits can be realized using the described vascular grafts and associated methods. First, the vascular grafts are mechanically strong for suturing and withstand blood pressure. Second, the vascular grafts are able to be modified so that bioactive molecules can be incorporated to the vascular grafts in a controllable fashion. As a result, the described vascular grafts can have anticoagulant properties as well as desirable immune-regulatory characteristics. Third, the vascular graft can be produced without using complex equipment other than a standard laboratory. Fourth, the various vascular graft components can be obtained in the controlled lab conditions, which eliminates issues related to pathogen transmission from animal tissues and quality control.

Fifth, the vascular graft is completely biological and can fully integrate with the native tissue and undergo remodeling as native tissues. Sixth, the vascular graft does not contain living cells, which does not require stringent schedule plans or conditions for manufacturing, storage, and transportation. Seventh, vascular grafts mimic the micro-structure of the vessel walls to provide optimal mechanical properties and orientation cues for endothelialization and remodeling. Finally, the described methods to fabricate the vascular graft allow for precise quantitative and spatial control of anticoagulant agents such as heparin and heparin binding growth factors (e.g. VEGF) to maximize graft patency after implantation.

### BRIEF DESCRIPTION OF THE DRAWINGS

FIGS. 1A to 1D show the scheme of fabricating methacrylate-functionalized ECM (ECM-MA).

FIG. 2 shows heparin modifications to ECM and the fabrication of a multilayered TEVG.

FIG. 3 shows an illustration of Different Layers of Acellular TEVG. Section 1 shows dendrimer-involved end point attachment chemistry employed to increase the amount of conjugated heparin in the graft lumen while preserving heparin's AT-binding sites for anticoagulation activities. Section 2 shows methacrylated ECM nanofibers and methacrylated heparin form the interpenetrating structure that can be loaded with heparin-binding growth factors. Section 3 shows the ECM nanofibers and heparin will be photocrosslinked to combine different ECM layers and avoid delamination.

FIG. 4 shows an image of generated aligned 2-D hDF-ECM.

FIG. 5A shows the quantification of the amount of available carboxylic acid group with toluidine blue O on the ECM. FIG. 5B shows the quantification of the amount of available amine group with acid orange II on the ECM.

FIG. 6A shows NMR spectrum of the digested ECM and ECM-MA. Method I and II used ethanol and aqueous solutions, respectively. Method I demonstrated better control for preservation of the fibrous structure and thus Method I was utilized thereafter. FIG. 6B shows SEM images of the ECM's nanofibrous structure.

FIG. 7A shows NMR spectra of the functionalization of gelatin molecule, and FIG. 7B shows NMR spectra of the functionalization of heparin molecule.

FIG. 8A shows Hep-MA chemically linked to the Gel-MA hydrogel (Gel-MA/Hep-MA) and high stability of Hep-MA in the hydrogel compared with the physically mixed heparin (Gel-MA/Hep) indicated by toluidine blue O. FIG. 8B shows the leaching test curve. FIG. 8C shows Hep-MA chemically linked to the ECM-MA (ECM/Hep-MA.

FIGS. 9A, 9B, and 9C show views of layer-by-layer deposition and crosslink of Gel-MA, Gel-MA/Hep-MA, and Gel-MA to form a 3-D tubular gel.

FIG. 10A shows the fabricated TEVG with a 1.5 mm diameter mandrel. The cross-section of the tubular gel and three different layers are shown by toluidine blue O.

FIG. 10B, FIG. 10C, and FIG. 10D show the generated TEVG and the cross-section of the generated TEVG stained by toluidine blue O, demonstrating minimum delamination and the spatial distribution of the conjugated heparin to the TEVG.

FIG. 11 shows the TEVG section was examined by hematoxylin and eosin (H&E staining).

FIG. 12 shows the stress-strain curve of ECM/Gel-MA substitutes.

FIG. 13 shows the relative HUVEC cell numbers after 2-day culture on the hydrogels. “Control” represents the 10% w/v Gel-MA gel. Hep-MA indicates 10% w/v Gel-MA with 1% w/v Hep-MA and “Physical” indicates 10% w/v Gel-MA with physically mixed 1% w/v non-modified heparin.

FIG. 14A shows a graph displaying cell count of HUVECs cultured on regular coverslips, gelatin-MA conjugated ECM sheets, heparin-conjugated ECM sheet with VEGF, and heparin and VEGF incorporated ECM sheets. There was not a significant difference, according to ANOVA with P<0.05, if Gel-MA is excluded. FIG. 14B shows a graph of cell count of HUVECs cultured on gelatin-MA conjugated ECM sheets, heparin-conjugated ECM sheet with VEGF, and heparin and VEGF incorporated ECM sheets.

FIG. 15 shows images of in vivo cell culture on the heparin conjugated and VEGF incorporated ECM.

FIG. 16A shows the design of the hemocompatibility test of the anticoagulant property of ECM/Hep by using human whole blood. FIGS. 16B and 16C show images of the negative control material containing ECM and gelatin only. FIGS. 16D and 16E show images of ECM modified by Hep-MA. FIGS. 16F and 16G show images of the positive control material containing ECM modified by end-point attached Hep (EPA-Hep).

FIG. 17A shows in vivo rat aorta grafting (end-to-end anastomosis). FIGS. 17B and 17C show HE staining, exhibiting a clear lumen of the TEVG and a dense vessel wall and well-organized cellular structure 4-week post implantation in a Sprague Dawley (SD) rat abdominal aorta model. FIG. 17D shows the degrading ECM layers (red arrows) and Gel-MA remnants (yellow arrow) in the TEVG 4-week post implantation in the SD rat abdominal aorta model. FIGS. 17E, 17F, 17G, and 17H show the immunofluorescent von Willebrand Factor (vWF) staining 4-week post implantation in the SD rat abdominal aorta model, where vWF (red arrows) displays a complete lining of ECs along the TEVG lumen, α-SMA (green star) confirms the formation of dense SMC layers, and F4/80 (purple arrows) shows very mild immune reactions in the adventitia region.

### DETAILED DESCRIPTION

In an illustrative aspect, a vascular graft is provided. The vascular graft comprises i) one or more extracellular matrix compositions and ii) one or more hydrogels.

In an embodiment, the extracellular matrix composition comprises a fibroblast-secreted composition. In an embodiment, the extracellular matrix composition comprises a dermal fibroblast-secreted composition. In an embodiment, the extracellular matrix composition comprises a human dermal fibroblast-secreted composition. In an embodiment, the extracellular matrix composition comprises a smooth muscle cell-secreted composition. In an embodiment, the extracellular matrix composition is harvested from cultured cells. In an embodiment, the extracellular matrix composition comprises a nanofibrous composition.

In an embodiment, the extracellular matrix composition further comprises heparin. In an embodiment, the heparin is physically interpenetrated with the extracellular matrix composition. In an embodiment, the heparin is covalently linked to the extracellular matrix composition. In an embodiment, the heparin is covalently cross-linked to embed the natural extracellular matrix composition. In an embodiment, the extracellular matrix composition further comprises methacrylate.

In an embodiment, the extracellular matrix composition is functionalized. Functionalization of extracellular matrix is generally known in the art and, for example, can incorporate pendent groups (e.g., thiol, alkene, alkyne, azide, etc.) to the backbone of biomolecules in the extracellular matrix so that other molecules can be chemically linked to the extracellular matrix through those pendent groups without changing the main structure of the native extracellular matrix.

In an embodiment, the extracellular matrix composition is functionalized with methacrylate. In an embodiment, the extracellular matrix composition is functionalized with an alkene. In an embodiment, the extracellular matrix composition is functionalized with an alkyne. In an embodiment, the extracellular matrix composition is functionalized with a thiol. In an embodiment, the extracellular matrix composition is functionalized with an azide. In an embodiment, the extracellular matrix composition is functionalized via radiation. In an embodiment, the extracellular matrix composition is functionalized via light radiation. In an embodiment, the extracellular matrix composition is functionalized via a biocompatible crosslinking reaction. In an embodiment, the extracellular matrix composition is functionalized via a biocompatible conjugation reaction.

In an embodiment, the extracellular matrix composition comprises a thickness between 10 μm and 100 μm. In an embodiment, the extracellular matrix composition comprises a thickness between 10 μm and 20 μm. In an embodiment, the extracellular matrix composition comprises a thickness between 20 μm and 30 μm. In an embodiment, the extracellular matrix composition comprises a thickness between 30 μm and 40 μm. In an embodiment, the extracellular matrix composition comprises a thickness between 40 μm and 50 μm. In an embodiment, the extracellular matrix composition comprises a thickness of 15 μm.

In an embodiment, the hydrogel is a gelatin hydrogel. In an embodiment, the hydrogel further comprises heparin. In an embodiment, the hydrogel further comprises methacrylate.

In an embodiment, the hydrogel is functionalized. Functionalization of hydrogel is generally known in the art and, for example, can incorporate pendent groups (e.g., thiol, alkene, alkyne, azide, etc.) to synthetic or natural hydrogel molecules so that those hydrogel molecules can be crosslinked to form a three-dimensional (3D) network or be chemically linked to other molecules with the corresponding reactive groups.

In an embodiment, the hydrogel is functionalized with methacrylate. In an embodiment, the hydrogel is functionalized with an alkene. In an embodiment, the hydrogel is functionalized with an alkyne. In an embodiment, the hydrogel is functionalized with a thiol. In an embodiment, the hydrogel is functionalized with an azide. In an embodiment, the hydrogel is functionalized via a biocompatible crosslinking reaction. In an embodiment, the hydrogel is functionalized via a biocompatible conjugation reaction.

In an embodiment, the vascular graft comprises multiple layers. In an embodiment, the multiple layers comprise two or more extracellular matrix compositions. In an embodiment, the multiple layers comprise two or more hydrogels. In an embodiment, the multiple layers comprise two or more extracellular matrix compositions and two or more hydrogels.

In an embodiment, the vascular graft comprises one or more growth factors. In an embodiment, the growth factor is selected from the group consisting of fibroblast growth factor (FGF), vascular endothelial growth factor (VEGF), and epidermal growth factor (EGF), midkine, pleiotrophin, and any combination thereof. In an embodiment, the growth factor comprises vascular endothelial growth factor (VEGF). In an embodiment, the growth factor comprises fibroblast growth factor (FGF). In an embodiment, the growth factor comprises epidermal growth factor (EGF). In an embodiment, the growth factor comprises midkine. In an embodiment, the growth factor comprises pleiotrophin. In an embodiment, the growth factor comprises a heparin-binding growth factor.

In an embodiment, the vascular graft has a thickness between 100 μm and 1.5 mm. In an embodiment, the vascular graft has a thickness between 100 μm and 200 μm. In an embodiment, the vascular graft has a thickness between 200 μm and 300 μm. In an embodiment, the vascular graft has a thickness between 300 μm and 400 μm. In an embodiment, the vascular graft has a thickness between 400 μm and 500 μm. In an embodiment, the vascular graft has a thickness between 500 μm and 600 μm. In an embodiment, the vascular graft has a thickness between 600 μm and 700 μm. In an embodiment, the vascular graft has a thickness between 700 μm and 800 μm. In an embodiment, the vascular graft has a thickness between 800 μm and 900 μm. In an embodiment, the vascular graft has a thickness between 900 μm and 1000 μm.

In an embodiment, the vascular graft has a thickness between 1.0 mm and 1.1 mm. In an embodiment, the vascular graft has a thickness between 1.1 mm and 1.2 mm. In an embodiment, the vascular graft has a thickness between 1.2 mm and 1.3 mm. In an embodiment, the vascular graft has a thickness between 1.3 mm and 1.4 mm. In an embodiment, the vascular graft has a thickness between 1.4 mm and 1.5 mm.

In an embodiment, the vascular graft has a diameter between 750 μm and 6 mm. In an embodiment, the vascular graft has a diameter between 750 μm and 800 μm. In an embodiment, the vascular graft has a diameter between 800 μm and 900 μm. In an embodiment, the vascular graft has a diameter between 900 μm and 1000 μm. In an embodiment, the vascular graft has a diameter between 1.0 mm and 1.5 mm. In an embodiment, the vascular graft has a diameter between 1.5 mm and 2.0 mm. In an embodiment, the vascular graft has a diameter between 2.0 mm and 2.5 mm. In an embodiment, the vascular graft has a diameter between 2.5 mm and 3.0 mm. In an embodiment, the vascular graft has a diameter between 3.0 mm and 3.5 mm. In an embodiment, the vascular graft has a diameter between 3.5 mm and 4.0 mm. In an embodiment, the vascular graft has a diameter between 4.0 mm and 4.5 mm. In an embodiment, the vascular graft has a diameter between 4.5 mm and 5.0 mm. In an embodiment, the vascular graft has a diameter between 5.0 mm and 5.5 mm. In an embodiment, the vascular graft has a diameter between 5.5 mm and 6.0 mm.

In an embodiment, the vascular graft is substantially free of cells. In an embodiment, the vascular graft is substantially free of living cells. In an embodiment, the vascular graft is substantially free of nucleic acid. As used herein, the term “substantially free” refers to zero or nearly no detectable amount of a material, quantity, or item. For example, the amount can be less than 1 percent, less than 0.5 percent, less than 0.1 percent, or less than 0.01 percent of the material, quantity, or item. A vascular graft that is made from cell-derived extracellular matrix and substantially free of cells, living cells, and/or nucleic acid can be referred to as “completely biological.”

In an embodiment, the vascular graft is acellular. In an embodiment, the vascular graft is cylindrical shaped.

In an illustrative aspect, a method of forming a vascular graft is provided. The method comprises the step of combining an extracellular matrix composition and a hydrogel to form the vascular graft. The previously described embodiments of the vascular graft are applicable to the method of forming a vascular graft described herein.

In an embodiment, the method comprises combining multiple extracellular matrix and multiple hydrogels to form the vascular graft. In an embodiment, the step of combining comprises rolling the extracellular matrix composition and the hydrogel. In an embodiment, the rolling is performed via a mandrel, which is generally known to a person of ordinary skill in the art.

In an embodiment, the step of combining comprises wrapping the extracellular matrix composition and the hydrogel around a mandrel. In an embodiment, the step of combining comprises wrapping the extracellular matrix composition and the hydrogel around a nonadhesive mandrel. In an embodiment, the method forms a cylindrical vascular graft.

In an illustrative aspect, a method of administering a vascular graft to a patient in need thereof is provided. The method comprises the step of placing the vascular graft in the patient, wherein the vascular graft comprises i) one or more extracellular matrix compositions and ii) one or more hydrogels. The previously described embodiments of the vascular graft are applicable to the method of administering a vascular graft described herein.

In an embodiment, the method of administering is associated with a cardiac procedure on the patient. In an embodiment, the method of administering is associated with a reconstructive procedure. In an embodiment, the method of administering is associated with a vascular access procedure for hemodialysis.

Various embodiments of the invention are provided throughout the present disclosure. For instance, the following numbered embodiments are contemplated and are non-limiting:
1. A vascular graft comprising i) one or more extracellular matrix compositions and ii) one or more hydrogels.
2. The vascular graft of clause 1, any other suitable clause, or any combination of suitable clauses, wherein the extracellular matrix composition comprises a fibroblast-secreted composition.
3. The vascular graft of clause 1, any other suitable clause, or any combination of suitable clauses, wherein the extracellular matrix composition comprises a dermal fibroblast-secreted composition.
4. The vascular graft of clause 1, any other suitable clause, or any combination of suitable clauses, wherein the extracellular matrix composition comprises a human dermal fibroblast-secreted composition.
5. The vascular graft of clause 1, any other suitable clause, or any combination of suitable clauses, wherein the extracellular matrix composition comprises a smooth muscle cell-secreted composition.
6. The vascular graft of clause 1, any other suitable clause, or any combination of suitable clauses, wherein the extracellular matrix composition is harvested from cultured cells.
7. The vascular graft of clause 1, any other suitable clause, or any combination of suitable clauses, wherein the extracellular matrix composition comprises a nanofibrous composition.
8. The vascular graft of clause 1, any other suitable clause, or any combination of suitable clauses, wherein the extracellular matrix composition further comprises heparin.
9. The vascular graft of clause 8, any other suitable clause, or any combination of suitable clauses, wherein the heparin is physically interpenetrated with the extracellular matrix composition
10. The vascular graft of clause 8, any other suitable clause, or any combination of suitable clauses, wherein the heparin is covalently linked to the extracellular matrix composition.
11. The vascular graft of clause 8, any other suitable clause, or any combination of suitable clauses, wherein the heparin is covalently cross-linked to embed the natural extracellular matrix composition.
12. The vascular graft of clause 1, any other suitable clause, or any combination of suitable clauses, wherein the extracellular matrix composition further comprises methacrylate.
13. The vascular graft of clause 1, any other suitable clause, or any combination of suitable clauses, wherein the extracellular matrix composition is functionalized.
14. The vascular graft of clause 13, any other suitable clause, or any combination of suitable clauses, wherein the extracellular matrix composition is functionalized with methacrylate.
15. The vascular graft of clause 13, any other suitable clause, or any combination of suitable clauses, wherein the extracellular matrix composition is functionalized with an alkene.
16. The vascular graft of clause 13, any other suitable clause, or any combination of suitable clauses, wherein the extracellular matrix composition is functionalized with an alkyne.
17. The vascular graft of clause 13, any other suitable clause, or any combination of suitable clauses, wherein the extracellular matrix composition is functionalized with a thiol.
18. The vascular graft of clause 13, any other suitable clause, or any combination of suitable clauses, wherein the extracellular matrix composition is functionalized with an azide.
19. The vascular graft of clause 13, any other suitable clause, or any combination of suitable clauses, wherein the extracellular matrix composition is functionalized via radiation.
20. The vascular graft of clause 13, any other suitable clause, or any combination of suitable clauses, wherein the extracellular matrix composition is functionalized via light radiation.
21. The vascular graft of clause 13, any other suitable clause, or any combination of suitable clauses, wherein the extracellular matrix composition is functionalized via a biocompatible crosslinking reaction.
22. The vascular graft of clause 13, any other suitable clause, or any combination of suitable clauses, wherein the extracellular matrix composition is functionalized via a biocompatible conjugation reaction.
23. The vascular graft of clause 1, any other suitable clause, or any combination of suitable clauses, wherein the extracellular matrix composition comprises a thickness between 10 μm and 100 μm.
24. The vascular graft of clause 1, any other suitable clause, or any combination of suitable clauses, wherein the extracellular matrix composition comprises a thickness between 10 μm and 20 μm.
25. The vascular graft of clause 1, any other suitable clause, or any combination of suitable clauses, wherein the extracellular matrix composition comprises a thickness between 20 μm and 30 μm.
26. The vascular graft of clause 1, any other suitable clause, or any combination of suitable clauses, wherein the extracellular matrix composition comprises a thickness between 30 μm and 40 μm.
27. The vascular graft of clause 1, any other suitable clause, or any combination of suitable clauses, wherein the extracellular matrix composition comprises a thickness between 40 μm and 50 μm.
28. The vascular graft of clause 1, any other suitable clause, or any combination of suitable clauses, wherein the extracellular matrix composition comprises a thickness of 15 μm.
29. The vascular graft of clause 1, any other suitable clause, or any combination of suitable clauses, wherein the hydrogel is a gelatin hydrogel.
30. The vascular graft of clause 1, any other suitable clause, or any combination of suitable clauses, wherein the hydrogel further comprises heparin.
31. The vascular graft of clause 1, any other suitable clause, or any combination of suitable clauses, wherein the hydrogel further comprises methacrylate.
32. The vascular graft of clause 1, any other suitable clause, or any combination of suitable clauses, wherein the hydrogel is functionalized.
33. The vascular graft of clause 32, any other suitable clause, or any combination of suitable clauses, wherein the hydrogel is functionalized with methacrylate.
34. The vascular graft of clause 32, any other suitable clause, or any combination of suitable clauses, wherein the hydrogel is functionalized with an alkene.
35. The vascular graft of clause 32, any other suitable clause, or any combination of suitable clauses, wherein the hydrogel is functionalized with an alkyne.
36. The vascular graft of clause 32, any other suitable clause, or any combination of suitable clauses, wherein the hydrogel is functionalized with a thiol.
37. The vascular graft of clause 32, any other suitable clause, or any combination of suitable clauses, wherein the hydrogel is functionalized with an azide.
38. The vascular graft of clause 32, any other suitable clause, or any combination of suitable clauses, wherein the hydrogel is functionalized via a biocompatible crosslinking reaction.
39. The vascular graft of clause 32, any other suitable clause, or any combination of suitable clauses, wherein the hydrogel is functionalized via a biocompatible conjugation reaction.
40. The vascular graft of clause 1, any other suitable clause, or any combination of suitable clauses, wherein the vascular graft comprises multiple layers.
41. The vascular graft of clause 40, any other suitable clause, or any combination of suitable clauses, wherein the multiple layers comprise two or more extracellular matrix compositions.
42. The vascular graft of clause 40, any other suitable clause, or any combination of suitable clauses, wherein the multiple layers comprise two or more hydrogels.
43. The vascular graft of clause 40, any other suitable clause, or any combination of suitable clauses, wherein the multiple layers comprise two or more extracellular matrix compositions and two or more hydrogels.
44. The vascular graft of clause 1, any other suitable clause, or any combination of suitable clauses, wherein the vascular graft comprises one or more growth factors.
45. The vascular graft of clause 44, any other suitable clause, or any combination of suitable clauses, wherein the growth factor is selected from the group consisting of fibroblast growth factor (FGF), vascular endothelial growth factor (VEGF), and epidermal growth factor (EGF), midkine, pleiotrophin, and any combination thereof.
46. The vascular graft of clause 44, any other suitable clause, or any combination of suitable clauses, wherein the growth factor comprises vascular endothelial growth factor (VEGF).
47. The vascular graft of clause 44, any other suitable clause, or any combination of suitable clauses, wherein the growth factor comprises fibroblast growth factor (FGF).
48. The vascular graft of clause 44, any other suitable clause, or any combination of suitable clauses, wherein the growth factor comprises epidermal growth factor (EGF).
49. The vascular graft of clause 44, any other suitable clause, or any combination of suitable clauses, wherein the growth factor comprises midkine.
50. The vascular graft of clause 44, any other suitable clause, or any combination of suitable clauses, wherein the growth factor comprises pleiotrophin.
51. The vascular graft of clause 44, any other suitable clause, or any combination of suitable clauses, wherein the growth factor comprises a heparin-binding growth factor.
52. The vascular graft of clause 1, any other suitable clause, or any combination of suitable clauses, wherein the vascular graft has a thickness between 100 μm and 1.5 mm.
53. The vascular graft of clause 1, any other suitable clause, or any combination of suitable clauses, wherein the vascular graft has a thickness between 100 μm and 200 μm.
54. The vascular graft of clause 1, any other suitable clause, or any combination of suitable clauses, wherein the vascular graft has a thickness between 200 μm and 300 μm.
55. The vascular graft of clause 1, any other suitable clause, or any combination of suitable clauses, wherein the vascular graft has a thickness between 300 μm and 400 μm.
56. The vascular graft of clause 1, any other suitable clause, or any combination of suitable clauses, wherein the vascular graft has a thickness between 400 μm and 500 μm.
57. The vascular graft of clause 1, any other suitable clause, or any combination of suitable clauses, wherein the vascular graft has a thickness between 500 μm and 600 μm.
58. The vascular graft of clause 1, any other suitable clause, or any combination of suitable clauses, wherein the vascular graft has a thickness between 600 μm and 700 μm.
59. The vascular graft of clause 1, any other suitable clause, or any combination of suitable clauses, wherein the vascular graft has a thickness between 700 μm and 800 μm.
60. The vascular graft of clause 1, any other suitable clause, or any combination of suitable clauses, wherein the vascular graft has a thickness between 800 μm and 900 μm.
61. The vascular graft of clause 1, any other suitable clause, or any combination of suitable clauses, wherein the vascular graft has a thickness between 900 μm and 1000 μm.
62. The vascular graft of clause 1, any other suitable clause, or any combination of suitable clauses, wherein the vascular graft has a thickness between 1.0 mm and 1.1 mm.
63. The vascular graft of clause 1, any other suitable clause, or any combination of suitable clauses, wherein the vascular graft has a thickness between 1.1 mm and 1.2 mm.
64. The vascular graft of clause 1, any other suitable clause, or any combination of suitable clauses, wherein the vascular graft has a thickness between 1.2 mm and 1.3 mm.
65. The vascular graft of clause 1, any other suitable clause, or any combination of suitable clauses, wherein the vascular graft has a thickness between 1.3 mm and 1.4 mm.
66. The vascular graft of clause 1, any other suitable clause, or any combination of suitable clauses, wherein the vascular graft has a thickness between 1.4 mm and 1.5 mm.
67. The vascular graft of clause 1, any other suitable clause, or any combination of suitable clauses, wherein the vascular graft has a diameter between 750 μm and 6 mm.
68. The vascular graft of clause 1, any other suitable clause, or any combination of suitable clauses, wherein the vascular graft has a diameter between 750 μm and 800 μm.
69. The vascular graft of clause 1, any other suitable clause, or any combination of suitable clauses, wherein the vascular graft has a diameter between 800 μm and 900 μm.
70. The vascular graft of clause 1, any other suitable clause, or any combination of suitable clauses, wherein the vascular graft has a diameter between 900 μm and 1000 μm.
71. The vascular graft of clause 1, any other suitable clause, or any combination of suitable clauses, wherein the vascular graft has a diameter between 1.0 mm and 1.5 mm.
72. The vascular graft of clause 1, any other suitable clause, or any combination of suitable clauses, wherein the vascular graft has a diameter between 1.5 mm and 2.0 mm.
73. The vascular graft of clause 1, any other suitable clause, or any combination of suitable clauses, wherein the vascular graft has a diameter between 2.0 mm and 2.5 mm.
74. The vascular graft of clause 1, any other suitable clause, or any combination of suitable clauses, wherein the vascular graft has a diameter between 2.5 mm and 3.0 mm.
75. The vascular graft of clause 1, any other suitable clause, or any combination of suitable clauses, wherein the vascular graft has a diameter between 3.0 mm and 3.5 mm.
76. The vascular graft of clause 1, any other suitable clause, or any combination of suitable clauses, wherein the vascular graft has a diameter between 3.5 mm and 4.0 mm.
77. The vascular graft of clause 1, any other suitable clause, or any combination of suitable clauses, wherein the vascular graft has a diameter between 4.0 mm and 4.5 mm.
78. The vascular graft of clause 1, any other suitable clause, or any combination of suitable clauses, wherein the vascular graft has a diameter between 4.5 mm and 5.0 mm.
79. The vascular graft of clause 1, any other suitable clause, or any combination of suitable clauses, wherein the vascular graft has a diameter between 5.0 mm and 5.5 mm.
80. The vascular graft of clause 1, any other suitable clause, or any combination of suitable clauses, wherein the vascular graft has a diameter between 5.5 mm and 6.0 mm.
81. The vascular graft of clause 1, any other suitable clause, or any combination of suitable clauses, wherein the vascular graft is substantially free of cells.
82. The vascular graft of clause 1, any other suitable clause, or any combination of suitable clauses, wherein the vascular graft is substantially free of living cells.
83. The vascular graft of clause 1, any other suitable clause, or any combination of suitable clauses, wherein the vascular graft is substantially free of nucleic acid.
84. The vascular graft of clause 1, any other suitable clause, or any combination of suitable clauses, wherein the vascular graft is acellular.
85. The vascular graft of clause 1, any other suitable clause, or any combination of suitable clauses, wherein the vascular graft is cylindrical shaped.
86. A method of forming a vascular graft, said method comprising the step of combining an extracellular matrix composition and a hydrogel to form the vascular graft.
87. The method of clause 86, any other suitable clause, or any combination of suitable clauses, wherein the method comprises combining multiple extracellular matrix and multiple hydrogels to form the vascular graft.
88. The method of clause 86, any other suitable clause, or any combination of suitable clauses, wherein the step of combining comprises rolling the extracellular matrix composition and the hydrogel.
89. The method of clause 88, any other suitable clause, or any combination of suitable clauses, wherein the rolling is performed via a mandrel.
90. The method of clause 86, any other suitable clause, or any combination of suitable clauses, wherein the step of combining comprises wrapping the extracellular matrix composition and the hydrogel around a mandrel.
91. The method of clause 86, any other suitable clause, or any combination of suitable clauses, wherein the step of combining comprises wrapping the extracellular matrix composition and the hydrogel around a nonadhesive mandrel.
92. The method of clause 86, any other suitable clause, or any combination of suitable clauses, wherein the method forms a cylindrical vascular graft.
93. The method of clause 86, any other suitable clause, or any combination of suitable clauses, wherein the vascular graft is the vascular graft of any one of clauses 1-85.
94. A method of administering a vascular graft to a patient in need thereof, said method comprising the step of placing the vascular graft in the patient, wherein the vascular graft comprises i) one or more extracellular matrix compositions and ii) one or more hydrogels.
95. The method of clause 94, any other suitable clause, or any combination of suitable clauses, wherein the method of administering is associated with a cardiac procedure on the patient.
96. The method of clause 94, any other suitable clause, or any combination of suitable clauses, wherein the method of administering is associated with a reconstructive procedure.
97. The method of clause 94, any other suitable clause, or any combination of suitable clauses, wherein the method of administering is associated with a vascular access procedure for hemodialysis.
98. The method of clause 94, any other suitable clause, or any combination of suitable clauses, wherein the vascular graft is the vascular graft of any one of clauses 1-85.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Exemplary Objectives

The instant example provides objectives of the methods presented in Examples 2-8 described herein.

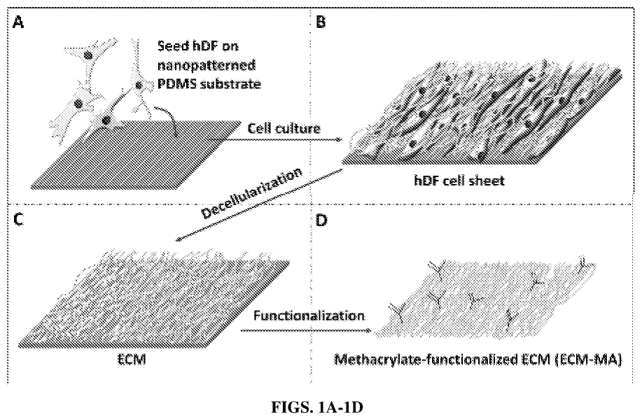

The present disclosure provides fabrication of biological tissue engineered vascular grafts (TEVGs) that contain morphological cues to induce specific cell orientation by using aligned nanofibrous materials developed from human dermal fibroblast-based extracellular matrix (hDF-ECM) and functionalized gelatin/heparin (Gel/Hep) hydrogels. In natural blood vessels, the smooth muscle cells (SMCs) and the reinforcing ECM fibers form an elastomotor helix inclined to the vessel centerline with 30-50° deviated from the vessel longitudinal axis. Thus, without being bound by any theory, a highly aligned nanofibrous natural ECM based acellular scaffold that mimics the specific orientation of fibrous structure within the native vessel might provide optimized mechanical property and facilitate the endothelialization and remodeling of the TEVG. A robust 2-D culture system to generate aligned hDF-ECM sheets and synthesized photo-initiated methacrylated gelatin (Gel-MA) and methacrylated heparin (Hep-MA) hydrogels that undergoes crosslinking controlled by light radiation was developed. After introducing methacrylate groups to the hDF-ECM (forming ECM-MA) (FIGS. 1A-1D), ECM sheets can be overlaid with controlled orientations to for a thicker 3-D constructs stabilized by hydrogels. The chemical and mechanical properties of the functionalized ECM and hydrogels and preliminary endothelialization analysis was performed.

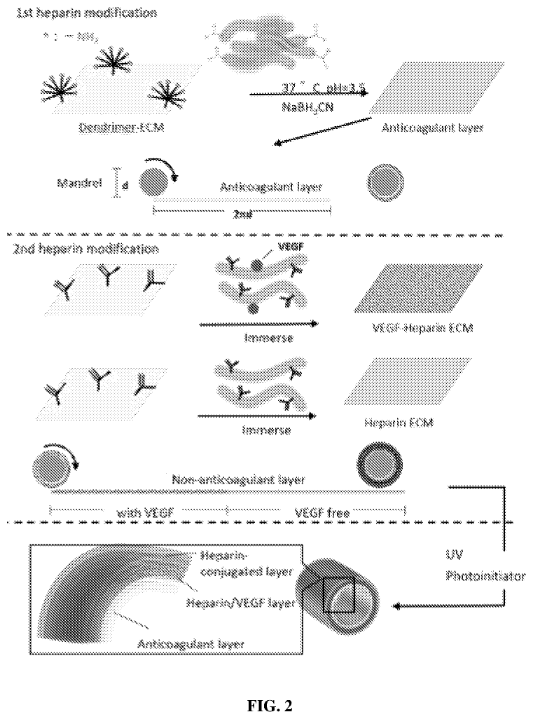

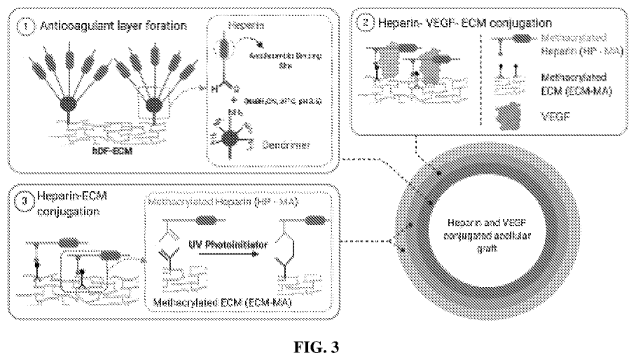

In general, functionalized ECM sheets are saturated in the hydrogels and wrapped around a mandrel with defined dimensions to form a tubular cellular assembly. Specifically, end-point-attached heparin is conjugated to the innermost layer for anticoagulant purposes. Methacrylated heparin (Hep-MA) and growth factors (e.g. vascular endothelial growth factor, VEGF) are incorporated to the mid layer to attract and direct immune cells to infiltrate and differentiate. Hep-MA is conjugated to the outer layer. Different layers are combined with methacrylated gelatin (Gel-MA) that can covalently bind to the methacrylated hDF-ECM (ECM-MA) base material through a controlled photo-initiated reaction (FIGS. 2 and 3).

### Example 2

### Generation of Aligned hDF-ECM

The primary material for fabrication of the acellular tissue engineered vascular graft (TEVG) includes highly aligned nanofibrous extracellular matrix (ECM) scaffold harvested from decellularizing the aligned human cell sheet, e.g. the human dermal fibroblast (hDF) cell sheet. The fibrous structure within the hDF-ECM scaffold has a similar size as natural collagen nanofibers of native tissues (80 nm), and is also rich in elastin, collagen and other biological materials that form the structural and functional components of vascular tissues. The hDF-ECM can be further functionalized with methacrylate groups for drug or bioactive molecule conjugation without compromising the nanofibrous structure.

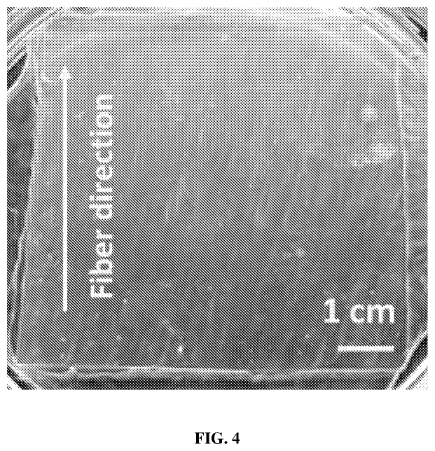

In the instant example, nanopatterned PDMS substrates (grated to 280 nm in depth, 350 nm in width, and a 700 nm periodicity) were used to culture hDF at a starting density of 10,000 cells/cm² for 8 weeks (FIG. 1A). Then, the fibroblast cell sheets were decellularized. The direction of the fiber can be observed on the generated hDF-ECM directly (FIG. 4).

### Example 3

### Development of the ECM-MA

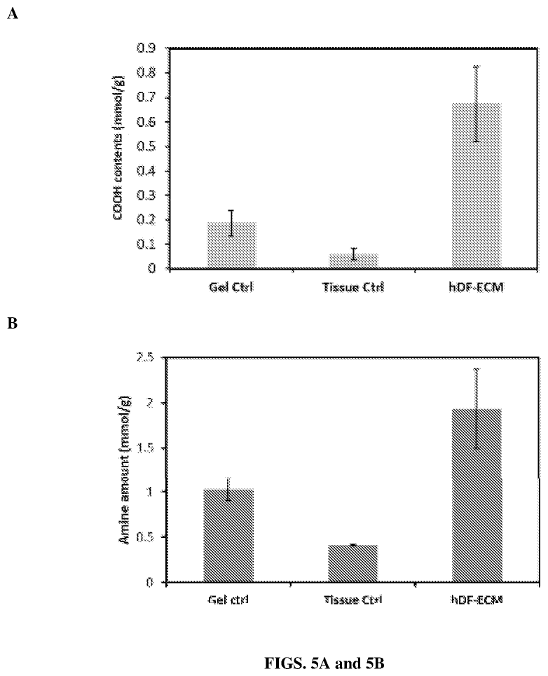

In the instant example, toluidine blue O and acid orange II were used to quantify the amount of available carboxylic acid group and amine group on the ECM, respectively (FIGS. 5A and 5B). The methacrylate functionalization methods are performed through amine or carboxylic acid groups. Without being bound by any theory, the results could indicate hDF-ECM contains a large number of active sites that can be potentially converted to methacrylate groups for molecular conjugation and reaction with hydrogels.

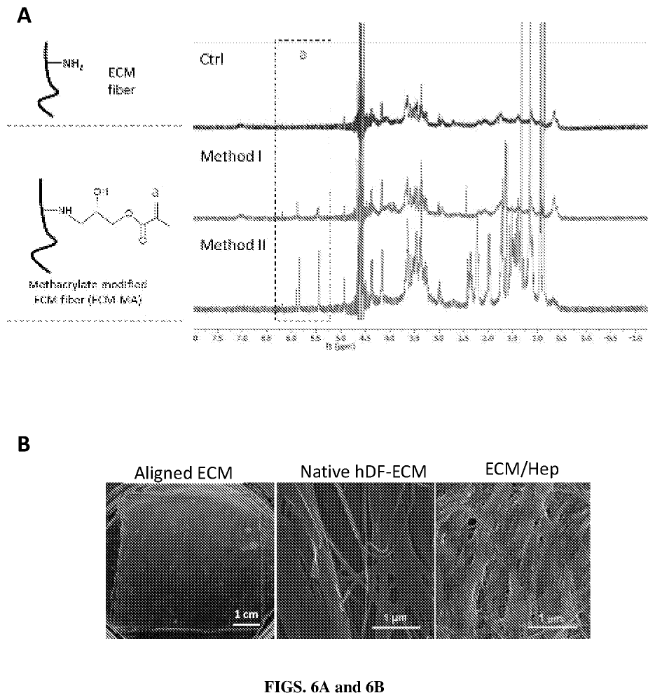

In the instant example, methacrylate groups were grafted to the hDF-ECM structure without damaging its nanofibour structure to introduce functional groups that may connect different ECM layers. Two glycidyl methacrylate (GMA) based methods were developed and both methods conjugated methacrylate groups to the hDF. These results were ascertained by performing nuclear magnetic resonance (NMR) on the digested ECM-MA, where the typical two peaks between 5.0 and 6.0 may indicate the conjugated methacrylate (FIG. 6A). This modification method does not damage ECM's nanofibrous structure demonstrated by scanning electron microscope (FIG. 6B). Thus, without being bound by any theory, other molecules such as methacrylate- and thiol-modified molecules could be further grafted to the ECM through this site.

### Example 4

### Development of Gel-MA and Hep-MA

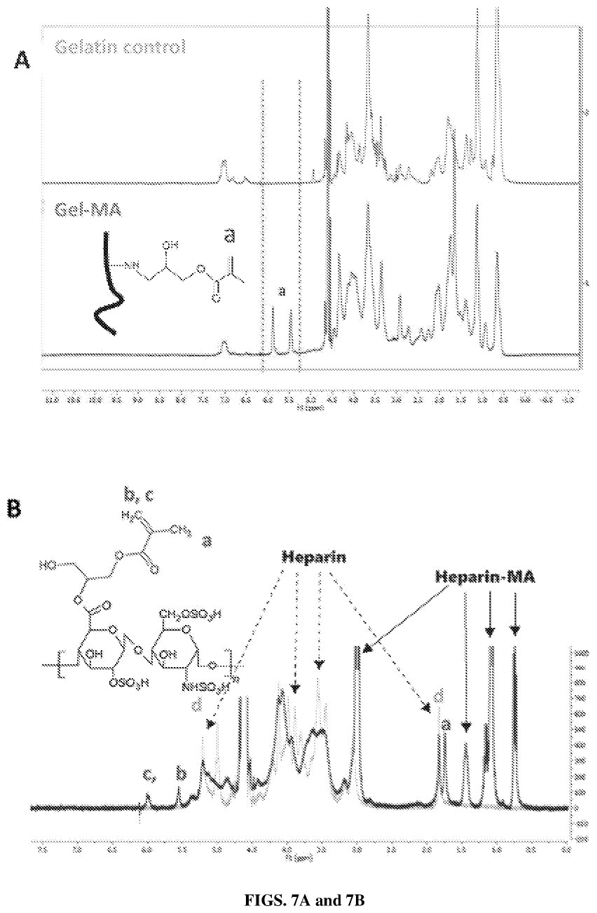

In the instant example, low amounts of photo-initiated hydrogel were used to combine different ECM-MA layers and stabilize the stacked 3-D structure. A biological gel, Gel-MA and Hep-MA, which may undergo controlled crosslinking was chosen. Gelatin is a protein derived from collagen, while heparin is an important glycosaminoglycan that can provide the TEVG with anti-coagulant properties and sequester growth factors for remodeling. The NMR data demonstrated the synthesis of Hep-MA and Gel-MA (FIGS. 7A and 7B). Gel-MA/Hep-MA gels were also prepared. Thus, both molecules can be grafted to the ECM-MA through reactions between methacrylate groups.

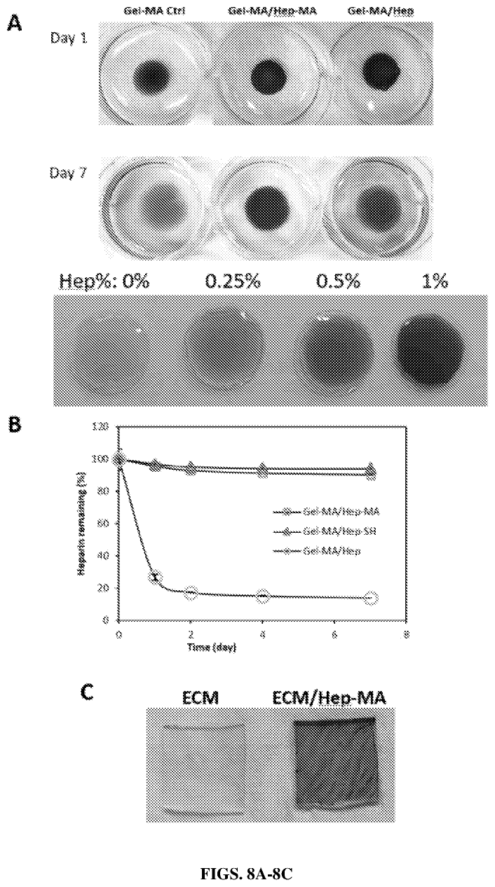

Light-induced reactions to incorporate a defined amount of Hep-MA to Gel-MA hydrogel were used. Then, the hydrogels were immersed into PBS solution and incubated at 37° C. for 7 days in total with PBS solution change every other day. The hydrogels were stained by TBO dye afterwards, where purple indicates the presence of heparin and the darkness of purple indicates the amount of heparin within the hydrogel (FIG. 8A). Quantitative analysis demonstrated over 90% of heparin remained in the hydrogel after 7-day in vitro incubation (FIG. 8B). Without being bound by any theory, these results may indicate the stability of the conjugated heparin. This reaction was also applied to ECM-MA to incorporate heparin to ECM (FIG. 8C).

### Example 5

### Fabrication of the Acellular Vascular Graft

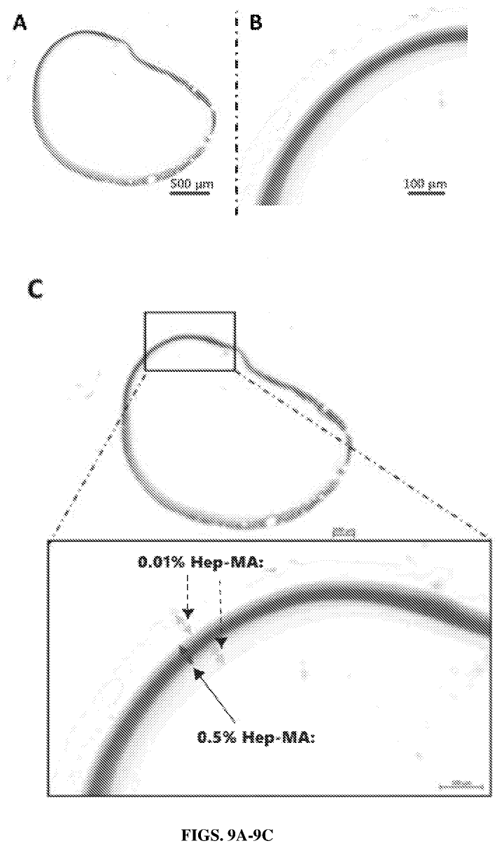

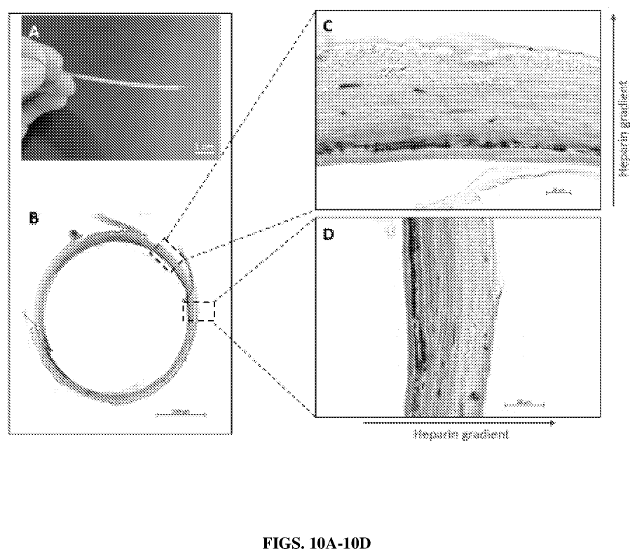

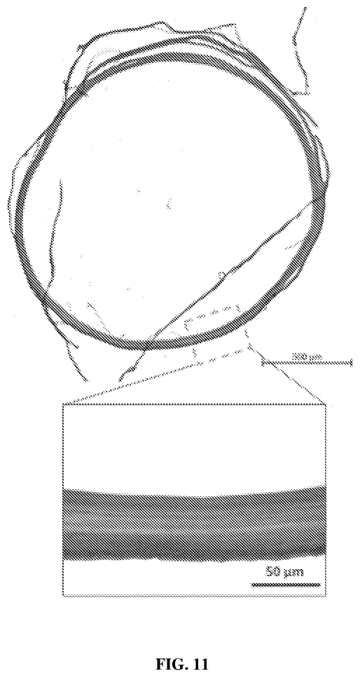

In the instant example, the fabrication is demonstrated through layer-by-layer conjugation and hydrogel crosslinking (FIGS. 9A-9C). The ECM-MA sheet was saturated in the 0.1% w/v Hep-MA precursor solutions to conjugate Hep-MA to the ECM structure. Then, the produced ECM-Hep sheets were soaked in another precursor solution (1% w/v Gel-MA or 1% Gel-MA/0.1% Hep-MA w/v) and wrapped to a 1.5 mm diameter mandrel. The ECM sheets were overlaid in such a way that the orientation of the ECM fiber was controlled to be +30 degree and −30 degree alternatively to the longitudinal direction of the vessel. Once all the layers were observed to be physically tight, the mandrel was exposed under 405 nm blue light for crosslinking (FIG. 10A). The fabricated TEVG was cross-sectioned and stained with toluidine blue O (TBO) dye, demonstrating the spatial distribution of the conjugated heparin to the TEVG (FIGS. 10B-10D). The TEVG section was examined by hematoxylin and eosin (H&E staining) (FIG. 11). ECM layers were fused together by hydrogels with minimum delamination.

### Example 6

### Preliminary Analysis of Mechanical Properties

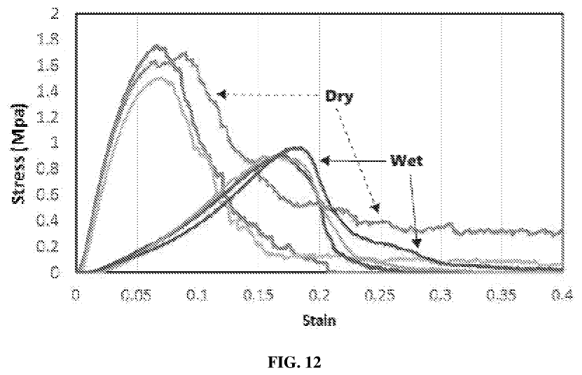

In the instant example, a fiber-gel composite structure as an ECM/Gel-MA substitute for the mechanical test was prepared. Cellulose fiber sheets were saturated in the 1% w/v Gel-MA solution, and exposed to 405 nm light for gel crosslinking. The generated product was applied to the 8872 Instron system for a tensile test when hydrated, as well as air-dried. The stress-strain curves are demonstrated in FIG. 12, and the numeric results are summarized in Table 1. Without being bound by any theory, the results could indicate the hydration degree affects the major mechanical properties of the samples.

TABLE 1
Mechanical properties of the dry and wet ECM/Gel-MA substitute

| | Ultimate strain | Ultimate stress (MPa) | Modulus |
|---|---|---|---|
| Dry | 0.070 ± 0.003 | 1.988 ± 0.153 | 31.09 ± 3.2 |
| Wet | 0.158 ± 0.004 | 1.215 ± 0.222 | 5.81 ± 0.16 |

### Example 7

### Preliminary Test of Endothelialization

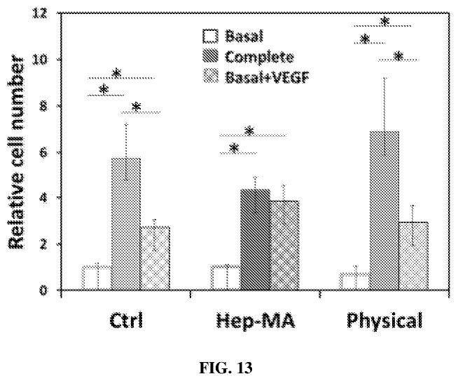

In the instant example, the growth of human umbilical vein endothelial cells (HUVECs) on the hydrogel system was examined to test the biocompatibility and the feasibility of using Hep-MA components to incorporate the vascular endothelial growth factor (VEGF) for TEVG endothelialization. Without being bound by any theory, the live-dead stain could indicate all the formulas are biocompatible. When VEGF was incorporated into the basal medium, HUVEC demonstrated higher numbers after 2-day culture especially in the Hep-MA group, where the HUVEC cell number was essentially equal to the group where the complete culture medium is used (FIG. 13).

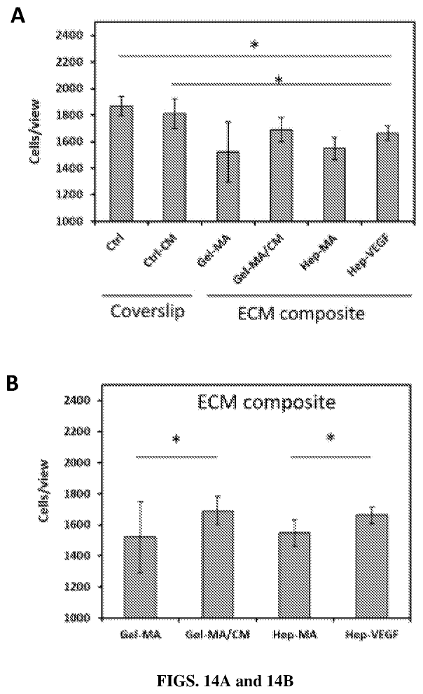

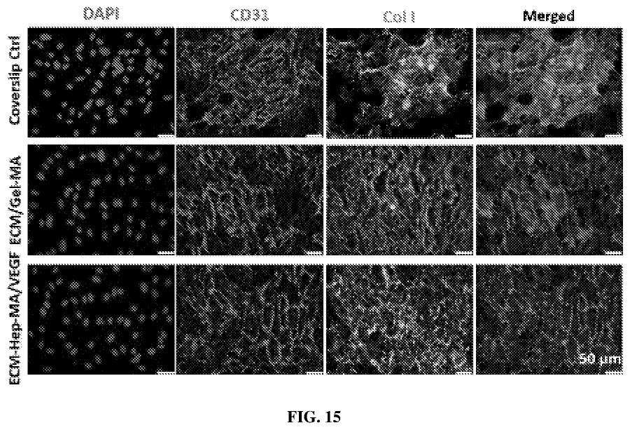

The heparin-conjugated ECM sheet with VEGF solution (500 ng/mL) were soaked for 24 hours, where VEGF can bind to heparin non-covalently. Then, the heparin and VEGF incorporated ECM sheets were used to culture the human umbilical vein endothelial cell (HUVEC) for 3 days with a starting cell density of 20,000 cell/cm². HUVECs cultured on regular coverslips and gelatin-MA conjugated ECM sheets were used as controls. Without being bound by any theory, the results could indicate the biocompatibility of the modified ECM sheets, where no cytotoxicity was observed and HUVEC took orientation along with the aligned ECM. Quantitative analysis shows that with the incorporated VEGF, HUVEC number enhanced by 7.3% after 3 days (FIGS. 14A and 14B). In vivo cell culture on the heparin-conjugated and VEGF incorporated ECM was also performed (FIG. 15).

### Example 8

### In Vitro Hemocompatibility Test

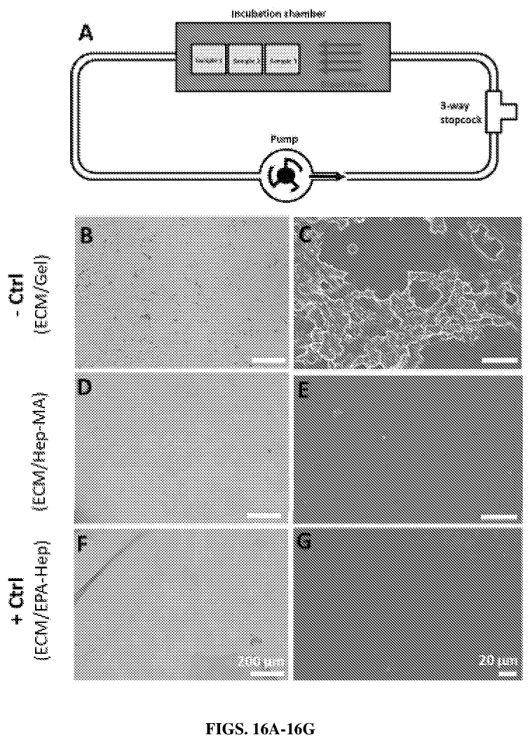

In the instant example, anticoagulant properties of ECM/Hep were analyzed by using human whole blood and perfusion chamber in vitro (FIG. 16A). Without being bound by any theory, this 2D model demonstrated that TEVG material (heparin conjugated ECM) may prevent blood cells from adhering. The control material contained ECM and gelatin only, and showed significant platelet adhesion (FIGS. 16B and 16C). ECM modified by Hep-MA and end-point attached Hep (EPA-Hep) demonstrated excellent anticoagulant properties (FIG. 16D to 16G). The incubation in the perfusion chamber lasted for 30 minutes.

### Example 9

### In Vivo Animal Test

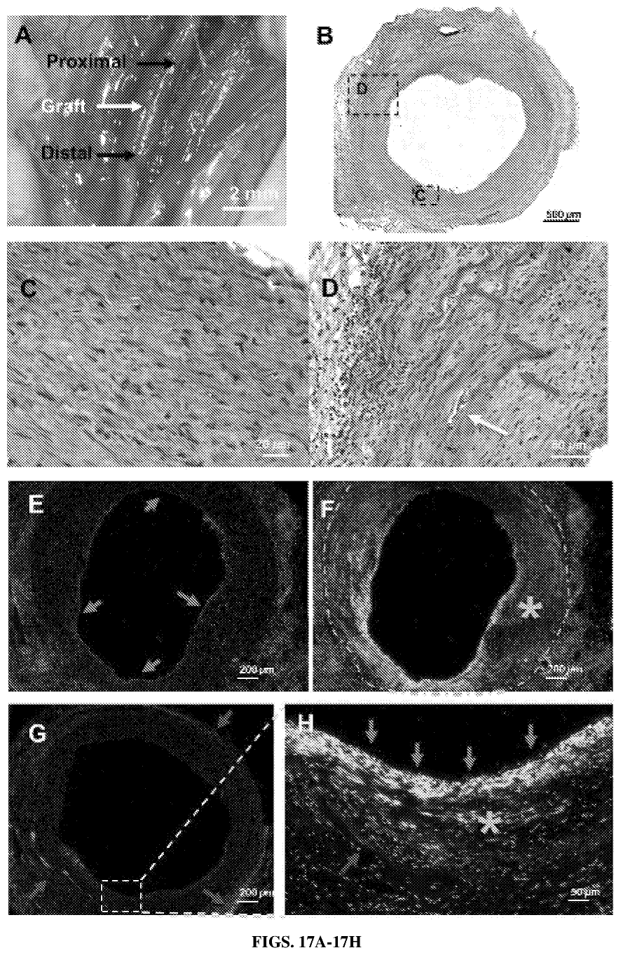

In the instant example, an acellular TEVG made from ECM, Gel-MA, Hep-MA was pre-soaked in 500 ng/mL VEGF for 24 hours and then transplanted into the SD rat abdominal aorta for 4 weeks (see FIG. 17A). No exogenous anticoagulant agent was administered after the surgery.

Excellent patency and desirable integration of the acellular TEVG were observed via host cell recruitment and graft remodeling (FIGS. 17B and 17C). After 4 weeks, the ECM layers (FIG. 17D; top three arrows) and Gel-MA remnants (FIG. 17D; bottom arrow) were observed in the extraluminal side.

The acellular TEVG were replaced with multiple layers of human α-smooth muscle actin (α-SMA) positive cells, confirming the recruitment of smooth muscle cells (SMAs) (FIG. 17F; asterisk). There was also a lining of rat von Willebrand factor (vWF)-positive cells along the luminal surface of the TEVG (FIG. 17E; arrows), suggesting the TEVG surface had recruited and been covered by ECs.

General macrophage marker F4/80 was observed mostly in the adventitia regions (see FIG. 17G; arrows and FIG. 17H; lowermost arrow), whereas few cells in the graft showed positive staining, indicating an absence of strong inflammatory reactions against the TEVG.

1. A vascular graft comprising i) one or more extracellular matrix compositions and ii) one or more hydrogels.

2. The vascular graft of claim 1, wherein the extracellular matrix composition comprises a fibroblast-secreted composition.

3. The vascular graft of claim 1, wherein the extracellular matrix composition comprises a dermal fibroblast-secreted composition.

4. The vascular graft of claim 1, wherein the extracellular matrix composition comprises a human dermal fibroblast-secreted composition.

5. The vascular graft of claim 1, wherein the extracellular matrix composition comprises a smooth muscle cell-secreted composition.

6. The vascular graft of claim 1, wherein the extracellular matrix composition comprises a nanofibrous composition.

7. The vascular graft of claim 1, wherein the extracellular matrix composition further comprises heparin.

8. The vascular graft of claim 7, wherein the heparin is physically interpenetrated with the extracellular matrix composition, or wherein the heparin is covalently linked to the extracellular matrix composition.

9. The vascular graft of claim 1, wherein the extracellular matrix composition further comprises methacrylate.

10.-15. (canceled)

16. The vascular graft of claim 1, wherein the vascular graft comprises multiple layers and wherein the multiple layers comprise two or more extracellular matrix compositions.

17. The vascular graft of claim 1, wherein the vascular graft comprises multiple layers and wherein the multiple layers comprise two or more hydrogels.

18. The vascular graft of claim 1, wherein the vascular graft comprises multiple layers and wherein the multiple layers comprise two or more extracellular matrix compositions and two or more hydrogels.

19.-21. (canceled)

22. The vascular graft of claim 1, wherein the vascular graft is substantially free of cells.

23. The vascular graft of claim 1, wherein the vascular graft is substantially free of living cells.

24. The vascular graft of claim 1, wherein the vascular graft is substantially free of nucleic acid.

25. The vascular graft of claim 1, wherein the vascular graft is acellular.

26.-31. (canceled)

32. A method of administering a vascular graft to a patient in need thereof, said method comprising the step of placing the vascular graft in the patient, wherein the vascular graft comprises i) one or more extracellular matrix compositions and ii) one or more hydrogels.

33. The method of claim 32, wherein the method of administering is associated with a cardiac procedure on the patient.

34. The method of claim 32, wherein the method of administering is associated with a reconstructive procedure.

35. The method of claim 32, wherein the method of administering is associated with a vascular access procedure for hemodialysis.

36. (canceled)
